(19) United States
(12) Patent Application Publication
(10) Pub. No.: US 2013/0309719 A1
(43) Pub. Date: Nov. 21, 2013

(54) HETEROTROPHIC MICROBIAL PRODUCTION OF XANTHOPHYLL PIGMENTS

(71) Applicant: Hywel David Griffiths, Auckland (NZ)

(72) Inventor: Hywel David Griffiths, Auckland (NZ)

(73) Assignee: PHOTONZ CORPORATION LIMITED, WAITAKERE (NZ)

(21) Appl. No.: 13/877,402

(22) PCT Filed: Oct. 6, 2011
(86) PCT No.: PCT/NZ11/00210

§ 371 Date: May 31, 2013

(30) Foreign Application Priority Data
Oct. 6, 2010 (NZ) 588418

(51) Int. Cl. C12P 23/00; C12N 1/12

(52) U.S. Cl. CPC C12P 23/00; C12N 1/12
USPC 435/67; 435/257.1

(57) ABSTRACT

The invention relates to a microbial biomass wherein the biomass comprises at least one xanthophyll selected from any one or more of fucoxanthin, diatoxanthin and diadinoxanthin. The invention further relates to a process for producing such a microbial biomass. Further the invention relates to compositions comprising at least one xanthophyll selected from any one or more of fucoxanthin, diatoxanthin and diadinoxanthin and processes for producing such compositions.

### FIELD OF INVENTION

The invention relates to a microbial biomass wherein the biomass comprises at least one xanthophyll selected from any one or more of fucoxanthin, diatoxanthin and diadinoxanthin. The invention further relates to a process for producing such a microbial biomass. Further the invention relates to compositions comprising at least one xanthophyll selected from any one or more of fucoxanthin, diatoxanthin and diadinoxanthin and processes for producing such compositions.

### BACKGROUND

Xanthophylls are a class of oxygenated carotenoids containing long series of conjugated double bonds. They are generally yellow to red in colour and are produced by organisms often as accessory pigments to fermentation. Their structures also make them good antioxidants.

Several xanthophylls have value as colourants and nutritional supplements and interest is growing in their therapeutic use.

The xanthophyll fucoxanthin (Fx) has been demonstrated to stimulate reduction of white adipose tissue in mice (Maeda et al. Biochem Biophys Res Commun. 332(2): 392-7, 2005) and to increase resting energy expenditure in obese, non-diabetic female volunteers with non-alcoholic fatty liver disease (Abidov at al. Diabetes, Obesity and Metabolism 12: 72-81, 2010). As a result it is already widely used as a weight-loss supplement. It is also attracting attention for its effects on lipid metabolism (Woo et al. Chemico-Biological Interactions, 186, 316-322, 2010) and anti-carcinogenic effects (Kotake-Nara et al., J. Nutr, 131: 3303-3306,2001; Sugawara et al. J. Agric. Food Chem. 54: 9805-9810, 2006; Das et al. Biochim. Biophys. Acta, 1780: 743-749, 2008).

The xanthophyll diatoxanthin (Dtx) has been implicated as an anti-inflammatory compound in studies on cell lines (Konishi at al. J. Oleo. Sci. 57(3): 181-9 2008). Diadinoxanthin (Ddx) is interconverted with Dtx by epoxidation/de-epoxidation and may also be of therapeutic value. Fucoxanthin is currently produced from macroalgae such as Laminaria japonica (kelp), Undaria pinnatifida (wakame) and Petalonia binghamiae (haba-nori). These seaweeds grow in the seas around Asia, often in a farmed process where young seaweeds are grown in tanks and then transplanted to the ocean floor once they are mature enough to survive. The mature plants are then harvested by machine and dried by hot air. Once harvested, the raw seaweed is processed to decrease the particle size (either by milling or cutting) before extraction with solvent (often ethanol) followed by column chromatography to separate Fx from other pigments, for example, chlorophylls (Kanazawa et al. Food Sci. Technol. Res., 14(6): 573-582 2008).

There are a number of disadvantages with this process. The seaweeds are exposed to environmental contaminants during their growth and the Fx thus produced must be monitored for substances such as arsenic. The seaweeds contain relatively low levels of Fx (below 0.1% of fresh weight) and require processing before extraction can be carried out (Kanazawa et al. 2008; Mori et al Mar. Drug 2: 63-72 2004). In addition, the areas where these seaweeds can be farmed are limited.

The Dtx used in the Konishi et al study was isolated from the sea squirt Halocynthia roretzi. Harvesting of wild sea squirts to provide Dtx also posses problems as any attempt at farming would require careful attention to their feed since the sea squirts do not make the xanthophylls themselves but take them in from their diet.

Thus, there is a need in the art to provide a method of production of xanthophyll pigments such as Fx, Dtx and Ddx which can produce a plentiful supply, is amenable to purification and extraction of the pigments and does not contain harmful contaminants. There is also a need for xanthophyll compositions that contain Fx, Dtx and Ddx in useful amounts.

It is an object of the invention to provide a microbial biomass comprising at least one xanthophyll selected from fucoxanthin, diatoxanthin and diadinoxanthin.

It is an alternative object of the invention to provide a method of producing a biomass through heterotrophic fermentation, said biomass comprising at least one xanthophyll selected from fucoxanthin, diatoxanthin and diadinoxanthin, and to provide a biomass so produced.

It is a further alternative object of the invention to provide a xanthophyll composition comprising at least one xanthophyll selected from fucoxanthin, diatoxanthin and diadinoxanthin.

It is a further alternative object of the invention to provide an enriched xanthophyll composition comprising at least one xanthophyll selected from fucoxanthin, diatoxanthin and diadinoxanthin.

It is a further alternative object of the invention to provide a process for producing xanthophyll composition comprising at least one xanthophyll selected from fucoxanthin, diatoxanthin and diadinoxanthin.

Alternatively, it is an object of the invention to at least provide a useful choice to the public.

### SUMMARY OF THE INVENTION

According to a first aspect of the invention, there is provided a microbial biomass, produced from a heterotrophic fermentation, the biomass comprising at least one xanthophyll selected from any one or more of fucoxanthin, diatoxanthin and diadinoxanthin.

Preferably, the xanthophyll is fucoxanthin.

Preferably, the xanthophyll is diatoxanthin.

Preferably, the xanthophyll is diadinoxanthin.

Preferably, the amount of the xanthophyll in the biomass is quantified.

Preferably, the xanthophyll is present at levels equal to or greater than about 0.1% of dry cell weight.

Preferably, the xanthophyll is present at levels equal to or greater than about 0.2% of dry cell weight.

Preferably, the xanthophyll is present at levels equal to or greater than about 0.5% of dry cell weight.

Preferably, the xanthophyll is present at levels equal to or greater than about 1% of dry cell weight.

Preferably, the microbial biomass is a eukaryotic microbial biomass.

Preferably, the microbial biomass is a microalgal biomass.

Preferably, the microbial biomass is a marine diatom biomass.

Preferably, the microbial biomass is a marine single-celled diatom biomass.

Preferably, the microbial biomass is a Nitzschia biomass.

Preferably, the microbial biomass is a Nitzschia laevis biomass.

According to a second aspect of the invention, there is provided a process for producing a microbial biomass comprising at least one xanthophyll selected from any one or more of fucoxanthin, diatoxanthin and diadinoxanthin, the process comprising the steps of:
cultivating a microorganism in heterotrophic culture to produce the biomass; and
recovering said biomass.

Preferably, the xanthophyll is fucoxanthin.

Preferably, the xanthophyll is diatoxanthin.

Preferably, the xanthophyll is diadinoxanthin.

Preferably, the xanthophyll is present at levels equal to or greater than about 0.1% of dry cell weight.

Preferably, the xanthophyll is present at levels equal to or greater than about 0.2% of dry cell weight of the biomass.

Preferably, the xanthophyll is present at levels equal to or greater than about 0.5% of dry cell weight of the biomass.

Preferably, the xanthophyll is present at levels equal to or greater than about 1% of dry cell weight of the biomass.

Preferably, the xanthophyll is present at levels equal to or greater than about 5% of dry cell weight of the biomass.

Preferably, the xanthophyll is present at levels equal to or greater than about 8% of dry cell weight of the biomass.

Preferably, the fucoxanthin is present at levels equal to or greater than about 1% of dry cell weight of the biomass.

Preferably, the microorganism is eukaryotic.

Preferably, the microorganism is microalgal.

Preferably, the microorganism is a marine diatom biomass.

Preferably, the microorganism is a marine single-celled diatom.

Preferably, the microorganism is of the genus Nitzschia.

Preferably, the microorganism is Nitzschia laevis.

Preferably, the microorganism is selected for a capability of heterotrophic growth.

Preferably, the microorganism is selected or modified for a desired capability to produce at least one xanthophyll selected from any one or more of fucoxanthin, diatoxanthin and diadinoxanthin.

Preferably at least about 0.1 mg of the xanthophyll is produced per litre of culture per hour.

Preferably at least about 0.5 mg of the xanthophyll is produced per litre of culture per hour.

Preferably at least about 1 mg of the xanthophyll is produced per litre of culture per hour.

Preferably at least about 5 mg of the xanthophyll is produced per litre of culture per hour.

Preferably at least about 10 mg of the xanthophyll is produced per litre of culture per hour.

Preferably at least about 5 mg of the fucoxanthin is produced per litre of culture per hour.

Preferably, the step of cultivating the microorganism in heterotrophic culture comprises a culture phase in which cells are grown under conditions in which organic carbon is used as an energy source.

Preferably, the step of cultivating the microorganism in heterotrophic culture comprises a culture phase in which cells are grown under conditions of limitation of nutrients.

Preferably, the nutrients subject to limitation are selected from any one or more of phosphorus, nitrogen and/or silicon.

Preferably, the step of cultivating a microorganism in heterotrophic culture comprises exposing the culture to low levels of light.

Preferably, the step of cultivating a microorganism is carried out in a batch fermentation.

Alternatively, the step of cultivating a microorganism is carried out in a fed batch fermentation.

Alternatively, the step of cultivating a microorganism is carried out as a continuous fermentation.

Preferably, the step of cultivating a microorganism in heterotrophic culture comprises two stages;
(i) a growth phase in which cells are grown under conditions in which organic carbon is used as an energy source and other nutrients are not limiting, said first step being undertaken to accumulate biomass, and
(ii) a finishing phase in which cells are grown under alternative conditions from the first stage.

Preferably, the alternative conditions in stage (ii) are selected from any one of more of: limitation of nutrients, changes in pH, changes in temperature, changes in salinity and/or exposure to low levels of light energy, said conditions being undertaken in order to maximise the amount of recoverable xanthophyll in the biomass.

Preferably, the pH range in stage (ii) is about 7 to 9.

Preferably, the temperature range in stage (ii) is about 15° C. to 40° C.

Preferably, the light levels of light energy in stage (ii) provide a maximum of about 10% of the energy supply for the culture.

Preferably, the nutrients subject to limitation in stage (ii) are selected from any one or more of phosphorus, nitrogen and silicon.

According to a third aspect of the invention, there is provided a microbial biomass produced by the process of the second aspect of this invention, the biomass comprising at least one xanthophyll selected from any one or more of fucoxanthin, diatoxanthin and diadinoxanthin.

Preferably, the xanthophyll is fucoxanthin.

Preferably, the xanthophyll is diatoxanthin.

Preferably, the xanthophyll is diadinoxanthin.

Preferably, the xanthophyll is present at levels greater than about 0.1% of dry cell weight.

Preferably, the xanthophyll is present at levels greater than about 0.5% of dry cell weight.

Preferably, the xanthophyll is present at levels greater than about 1% of dry cell weight.

Preferably, the xanthophyll is present at levels equal to or greater than about 5% of dry cell weight of the biomass.

Preferably, the xanthophyll is present at levels equal to or greater than about 8% of dry cell weight of the biomass.

Preferably, the fucoxanthin is present at levels equal to or greater than about 1% of dry cell weight of the biomass.

Preferably, the microbial biomass is a eukaryotic microbial biomass.

Preferably, the microbial biomass is a microalgal biomass.

Preferably, the microbial biomass is a marine diatom biomass.

Preferably, the microbial biomass is a marine single-celled diatom biomass.

Preferably, the microbial biomass is a Nitzschia biomass.

Preferably, the microbial biomass is a Nitzschia laevis biomass.

According to a fourth aspect of the invention, there is provided a xanthophyll composition comprising at least one xanthophyll selected from any one or more of fucoxanthin, diatoxanthin and diadinoxanthin, wherein the composition is extracted from a microbial biomass produced from a heterotrophic fermentation.

Preferably, the composition is extracted from the microbial biomass by selective or non-selective extraction.

Preferably, the solvent used for the non-selective extraction is selected from near-critical di-methyl ether, isopropanol and/or ethanol.

Preferably, the solvent used for the selective extraction is acetone.

Preferably, the xanthophyll is present at levels of about 0.1 to 60% by weight.

Preferably, the xanthophyll is present at levels of about 0.1 to 40% by weight.

Preferably, the xanthophyll is present at levels of about 0.5 to 40%, by weight.

Preferably, the xanthophyll is present at levels of about 1 to 40% by weight.

According to a fifth aspect of the invention, there is provided an enriched xanthophyll composition comprising at least one xanthophyll selected from any one or more of fucoxanthin, diatoxanthin and diadinoxanthin, wherein the composition is extracted from a microbial biomass produced from a heterotrophic fermentation and purified.

Preferably, the extraction is selective or non-selective extraction.

Preferably, the solvent used for the non-selective extraction is selected from near-critical di-methyl ether, isopropanol and/or ethanol.

Preferably, the solvent used for the selective extraction is acetone.

Preferably, the purification is carried out by chromatography.

Preferably, the xanthophyll is present at levels of at least about 5% by weight.

Preferably, the xanthophyll is present at levels of at least about 10% by weight.

Preferably, the xanthophyll is present at levels of at least about 20% by weight.

Preferably, the xanthophyll is present at levels of at least about 25% by weight.

Preferably, the xanthophyll is present at levels of at least about 60% by weight.

According to a sixth aspect of the invention, there is provided a process for producing a xanthophyll composition, wherein the xanthophyll is selected from any one or more of fucoxanthin, diatoxanthin and diadinoxanthin, the process comprising the steps of:
cultivating a microorganism in heterotrophic culture to produce a biomass;
recovering said biomass; and
extracting said xanthophyll composition from said biomass.

Preferably, the xanthophyll is fucoxanthin.

Preferably, the xanthophyll is diatoxanthin.

Preferably, the xanthophyll is diadinoxanthin.

Preferably, the biomass is a eukaryotic microbial biomass.

Preferably, the biomass is a microalgal biomass.

Preferably, the biomass is a marine diatom biomass.

Preferably, the biomass is a marine single-celled diatom biomass.

Preferably, the biomass is a Nitzschia biomass.

Preferably, the biomass is a Nitzschia laevis biomass.

Preferably, at least about 0.1 mg of the xanthophyll is produced per litre of culture per hour.

Preferably, at least about 0.5 mg of the xanthophyll is produced per litre of culture per hour.

Preferably at least about 1 mg of the xanthophyll is produced per litre of culture per hour.

Preferably at least about 5 mg of the xanthophyll is produced per litre of culture per hour.

Preferably at least about 10 mg of the xanthophyll is produced per litre of culture per hour.

Preferably at least about 5 mg of the fucoxanthin is produced per litre of culture per hour.

Preferably, the xanthophyll composition is recovered from the biomass by extraction.

Preferably, the process includes further includes the step of purification of the xanthophyll composition to produce an enriched xanthophyll composition.

According to a seventh aspect of the invention, there is provided a process for producing a xanthophyll composition derived from a process as described in the second aspect of this invention, wherein the xanthophyll is selected from any one or more of fucoxanthin, diatoxanthin and diadinoxanthin, and wherein the composition is obtained by a harvesting process comprising the steps of:
(a) harvesting cells from the heterotrophic culture;
(b) optionally, heating or otherwise killing the cells;
(c) forming the cells into a cake;
(d) optionally drying the cake to reduce or eliminate water content;
(e) extracting the cake with a non-selective or selective solvent and recovering the extracted material from the solvent as a residue to produce the xanthophyll composition;
(f) optionally further purifying the xanthophyll composition to produce an enriched xanthophyll composition.

Preferably, the non-selective solvent is selected from near-critical di-methyl ether, isopropanol and/or ethanol.

Preferably, the selective solvent is acetone.

Preferably, the optional further purification is carried out by chromatography.

According to a eighth aspect of the invention, there is provided a process for producing a xanthophyll composition, wherein the xanthophyll is selected from any one or more of fucoxanthin, diatoxanthin and diadinoxanthin, comprising the steps of:
cultivating a microorganism in heterotrophic culture to produce a biomass;
recovering said biomass;
extracting said biomass with solvent; and
recovering the extracted material from the solvent as a residue to produce said xanthophyll composition.

According to a ninth aspect of the invention, there is provided a process for producing an enriched xanthophyll composition, wherein the xanthophyll is selected from any one or more of fucoxanthin, diatoxanthin and diadinoxanthin, comprising the steps of:
cultivating a microorganism in heterotrophic culture to produce a biomass; recovering said biomass; and
recovering a xanthophyll composition from said biomass, and
purifying said xanthophyll composition to produce said enriched composition.

Preferably, the xanthophyll composition is recovered from the microbial biomass by extraction.

Preferably, the xanthophyll composition is recovered from the microbial biomass by selective or non-selective extraction.

Preferably, the solvent used for the non-selective extraction is selected from near-critical di-methyl ether and/or ethanol.

Preferably, the solvent used for the selective extraction is acetone.

Preferably, the purification is carried out by chromatography.

According to a tenth aspect of the invention, there is provided a process for producing an enriched xanthophyll composition, wherein the xanthophyll is selected from any one or more of fucoxanthin, diatoxanthin and diadinoxanthin, comprising the steps of:
cultivating a microorganism in heterotrophic culture to produce the biomass;
recovering said biomass;
extracting said biomass to produce a xanthophyll composition, and
purifying said xanthophyll composition to produce the enriched xanthophyll composition.

According to a eleventh aspect of the invention, there is provided a pharmaceutical or nutraceutical composition comprising the xanthophyll composition of the fourth aspect of the invention, together with a pharmaceutically acceptable excipient, wherein the xanthophyll is selected from any one or more of fucoxanthin, diatoxanthin and diadinoxanthin.

Preferably, the composition further includes lipids, fatty acids and/or fatty acid alkyl esters.

Preferably, the composition further includes plant and/or seed oil extracts.

According to a twelfth aspect of the invention, there is provided a pharmaceutical or nutraceutical composition comprising the xanthophyll composition of the fourth aspect of the invention or the enriched xanthophyll composition of the fifth aspect of the invention, together with a pharmaceutically acceptable excipient, wherein the xanthophyll is selected from any one or more of fucoxanthin, diatoxanthin and diadinoxanthin.

Preferably, the composition further includes lipids, fatty acids and/or fatty acid alkyl esters.

Preferably, the composition further includes plant and/or seed oil extracts.

Further aspects of the invention, which should be considered in all its novel aspects, will become apparent to those skilled in the art upon reading of the following description which provides at least one example of a practical application of the invention.

### BRIEF DESCRIPTION OF THE FIGURES

Embodiments of the invention will now be described, by way of example only, with reference to the accompanying drawings in which:

FIG. 1 a HPLC spectrum of an embodiment of the composition of the invention recorded at 449 nm

### DEFINITIONS AND ABBREVIATIONS

Fx means fucoxanthin which is a xanthophyll pigment.

Ddx means diadinoxanthin which is a xanthophyll pigment.

Dtx means diatoxanthin which is a xanthophyll pigment.

DCW, dry cell weight means the weight of a biomass once all water has been removed.

Heterotrophic culture means a culture of organisms for which at least 90% of the energy supply for the culture is derived from supplied nutrients which are usually a form or forms of organic carbon (e.g. glucose, acetate). Therefore a maximum of about 10% of the energy supply is derived from light energy. Preferably, less than 5% or less than 1% of the energy supply is derived from light energy. More preferably, the whole of the energy supply is from supplied nutrients.

Where there is reference to “absence” of light, this should be taken to mean substantially no light, but a person skilled in the art would be aware a small quantity of light is likely to be present at least periodically. In addition, a small quantity of light may be used to maximise component output as discussed herein.

### DETAILED DESCRIPTION OF THE INVENTION

Unexpectedly the present inventors have found that it is possible to obtain compositions including enriched compositions, containing or comprising a xanthophyll selected from any one or more of fucoxanthin, diatoxanthin and diadinoxanthin from microorganisms grown via heterotrophic fermentation.

An aspect of the present invention is the recognition that a biomass containing a xanthophyll selected from any one or more of fucoxanthin, diatoxanthin and diadinoxanthin can be produced via heterotrophic fermentation of microorganisms despite the lack of input of light.

The compositions and enriched compositions are capable of being produced through a process capable of producing quantities of a xanthophyll, selected from any one or more of fucoxanthin, diatoxanthin and diadinoxanthin, by extraction from a biomass produced via heterotrophic fermentation. The amount of xanthophyll produced has been found to be substantially sufficient for commercial use.

In a further embodiment, the invention also provides a microbial biomass produced from a heterotrophic fermentation, by the process of the first aspect, which comprises xanthophyll selected from any one or more of fucoxanthin, diatoxanthin and diadinoxanthin.

In a further embodiment, the invention provides a process or method for producing the microbial biomass comprising xanthophyll selected from any one of more of fucoxanthin, diatoxanthin and diadinoxanthin. The process or method comprises the steps of cultivating a microorganism in heterotrophic culture to produce the biomass and recovering said biomass.

Further, the microbial biomass produced from the heterotrophic fermentation comprises xanthophyll selected from any one or more of fucoxanthin, diatoxanthin and diadinoxanthin at levels greater than about 0.1% of dry cell weight, preferably greater than about 0.5% of dry cell weight, more preferably, greater than about 1% of dry cell weight, more preferably greater than about 5%, even more preferably greater than about 8% to a maximum of about 10% of dry cell weight (i.e. levels of xanthophyll in the biomass of about 0.1% to 10% of dry cell weight, preferably about 0.5% to 10% of dry cell weight, more preferably about 1% to 10% of dry cell weight, more preferably about 5% to 10%, more preferably about 8% to about 10%). Wherein the total dry cell weight of the xanthophyll is made up of one or more of the xanthophylls, selected from fucoxanthin, diatoxanthin and/or diadinoxanthin, either as a single compound or mixtures of two or three compounds. Xanthophylls other than fucoxanthin, diatoxanthin and/or diadinoxanthin may be present in the biomass, but these are not included in the calculation of the levels of xanthophyll for the purposes of this invention. Preferably, the fucoxanthin is present at levels equal to or greater than about 1% of dry cell weight of the biomass (i.e. levels between about 1% to about 10%).

Preferably the culture of microorganisms comprises identified microorganisms, or genetically modified microorganisms. Preferably the micro-organisms are eukaryotic, preferably microalgae. More preferably the microorganisms are marine diatoms, even more preferably the microorganisms are marine single-celled diatoms, preferably from the genus Nitzschia, even more preferably the micro-organisms are comprised of the marine single-celled diatom known as Nitzschia laevis. The microorganism is selected for its ability to produce at least one xanthophyll selected from fucoxanthin, diatoxanthin and/or diadinoxanthin. Examples of microorganisms capable of producing at least one of the desired xanthophyll include, but are not limited to members of the Diatomophyceae (such as Nitzschia laevis, Phaeodactylum tricomutum, or Cyclotella cryptica), members of the Prymnesiophyceae (such as Pavlova lutheri or Isochyrsis galbana) or members of the Pelagophyceae. The microorganism is also selected for its ability to grow under heterotrophic conditions. In some cases the microorganism could be genetically modified to either produce the xanthophyll pigment or to grow heterotrophically where it otherwise would not. A person skilled the art when provided with the disclosure in the present specification would be capable of selecting a microorganism capable of producing at least one of the xanthophyll selected from fucoxanthin, diatoxanthin and/or diadinoxanthin and capable of growth under heterotrophic conditions. The inventors have found the microorganism Nitzschia laevis to be suited for use in the invention as it still produces useful quantities of the xanthophyll pigments when grown in the absence of light and can be grown at sufficiently high cell density (in excess of 20 g dry cell weight per litre of culture) and growth rate (doubling times of less than 24 hours) to give high xanthophyll productivities.

The microorganisms can be genetically modified to contain a gene or series of genes that enable the organism to produce fucoxanthin, diatoxanthin and/or diadinoxanthin. Such genes could, for example, be identified from the completed genome sequences of the diatoms Thalassiosira pseudonana and Phaeodactylum tricomutum and sourced from publically available strains of either organism. The microorganism can also be selected, when under culture conditions, for an improved yield of recoverable fucoxanthin, diatoxanthin and/or diadinoxanthin.

The microorganism is cultured in a growth media comprising a chemical energy source, biologically assimilable nitrogen and phosphate, salts and minerals. Examples of suitable chemical energy sources include but are not limited to glucose, acetate, and ethanol. Examples of nitrogen sources include but are not limited to sodium nitrate, monosodium glutamate, yeast extract, urea or ammonia. Examples of phosphate sources include but are not limited to potassium-, dipotassium-, sodium-, or disodium-phosphates and phosphoric acid.

The quantity of chemical energy source should be provided in non-limiting quantities based on the amount of microorganism present. The rate of provision of this chemical energy source would depend upon the organism, type of source, growth rate of the organism and density of cells within the culture but could be determined by one skilled in the art when provided with this invention. The nitrogen and phosphate sources may optionally be provided in limiting quantities at certain phases of the growth of the organism to increase the production of one or more xanthophyll pigment.

The balance of salts and minerals required will be dependent on the microorganism selected. Likely salts and minerals required may include sodium, potassium, magnesium and calcium chlorides or sulphates, and salts of molybdenum, copper, zinc, cobalt, iron, nickel, selenium, and manganese. If the selected organism is a diatom, a source of silicate will also be necessary. A person skilled in the art would be capable of selecting a growth media suitable for the selected microorganism.

The growth media is regulated for aspects such as temperature, pH and dissolved oxygen content. These would be dependent on the microorganism selected but might be expected to lie in the range pH 7-9 (regulated by appropriate addition of either acid or base) 15-40° C. and dissolved oxygen of at least 20%. A dissolved oxygen content of greater than about 30% is preferred, more preferably greater than about or equal to about 40%. A person skilled in the art would be capable of selecting cultivation conditions suitable for the selected microorganism.

In an optional preferred aspect the process includes a culture phase in which cells are grown under conditions of limitation of nutrients, for example limitation of phosphorus, nitrogen, silicon and/or iron; said procedures being undertaken in order to maximise the amount of recoverable biomass and the content of fucoxanthin, diatoxanthin and/or diadinoxanthin therein. Such a maximisation process forms another aspect of the present invention.

Nutrient limitation means that the absence or low level of the nutrient in question causes the organism to grow more slowly than it would if the nutrient were present at higher levels or causes it to enter an alternative metabolic state, such as for instance, on in which carbon from the chemical energy source was used to generate lipids rather than proteins or nucleic acids.

Cultivation of the microorganism is carried out in a batch fermentation, or a fed batch fermentation, or carried out as a continuous fermentation.

The invention also provides a two step process for obtaining the biomass as previously described above wherein the process employs a culture of micro-organisms of a type selected for a capability of heterotrophic growth, and selected or modified for a capability of production of fucoxanthin, diatoxanthin and/or diadinoxanthin; the process including a first growth phase in which cells are grown under conditions in which organic carbon is used as an energy source and other nutrients are not limiting; said procedures being undertaken to accumulate biomass, and a second finishing phase in which cells are grown under alternative conditions; the alternative conditions including limitation of nutrients selected from a range including phosphorus, nitrogen and silicon, changes in pH, changes in temperature, changes in salinity and exposure to low levels of light energy; said procedures being undertaken in order to maximise the amount of recoverable fucoxanthin, diatoxanthin and/or diadinoxanthin in the biomass.

Preferred conditions for the second finishing phase include but are not limited to, pH in range of about 7 to 9 and temperature range of about 15° C. to 40° C.

If desired the cells may be exposed to low levels of light either broad spectrum or of particular wavelengths said procedures being undertaken in order to maximise the amount of recoverable fucoxanthin, diatoxanthin and/or diadinoxanthin in the biomass. Such light levels can be selected to provide a maximum of about 10% of the energy supply of for the culture, a level which will differ depending on the density, growth rate, and nutrient provision of the culture. The remaining energy supply for the culture being derived from supplied nutrients which are usually a form or forms of organic carbon, for example, glucose and/or acetate. Again such a maximisation process forms another aspect of the invention.

In a preferred option an average of at least about 0.1 mg fucoxanthin, diatoxanthin and/or diadinoxanthin is produced per litre of culture per hour. In a more preferred option at least about 0.5 mg fucoxanthin, diatoxanthin and/or diadinoxanthin is produced per litre of culture per hour. Reference to Example 3 herein shows that levels of above 5 mg per litre of culture per hour can be achieved and it is envisioned that levels of 10 mg per litre of culture per hour may be achieved.

Xanthophyll compositions containing any one of more of fucoxanthin, diatoxanthin and diadinoxanthin are preferably produced from the microbial culture by harvesting the microbes from the heterotrophic culture medium, optionally heating or otherwise killing the cells (for example to denature endogenous enzymes) and forming the cells into a biomass (e.g. a cake of biomass). Methods of killing the cells following harvesting would be known to a person skilled in the art, for example, one method would be to heat to temperatures in the region of 45-80° C., however precise temperatures would be dependent on the nature of the cells. An example of an alternative method of killing the cells would be lysis of the cells using, for example, rapid changes in pressure or enzymatic means. The biomass is optionally dried to reduce or eliminate water, methods of drying include, but are not limited to, freeze-drying, spray-drying, and refractance-window drying.

The microbial biomass of the invention, or produced by the process of the invention, may be subjected to one or more extraction steps to extract fucoxanthin, diatoxanthin and/or diadinoxanthin from the biomass to yield a xanthophyll composition (where the xanthophyll is selected from fucoxanthin, diatoxanthin and/or diadinoxanthin). Suitable extraction techniques are well known in the art. For example, the biomass may be extracted with a non-selective solvent which will dissolve the xanthophyll, such as near-critical di-methyl ether, ethanol or isopropanol, or a selective solvent such as acetone and the fucoxanthin, diatoxanthin and/or diadinoxanthin recovered from the solvent as a residue.

Following extraction, the level of fucoxanthin, diatoxanthin and/or diadinoxanthin in said composition is preferably at least 0.1%, preferably at least 0.5%, more preferably at least 1% Preferably the ranges of the level of fucoxanthin, diatoxanthin and/or diadinoxanthin in said composition are preferably from about 0.1 to 60%, preferably from about 0.1 to 40%, more preferably from about 0.5 to 40%, or even more preferably from about 1 to 40% by weight of the extracted material. Levels of about 15% to about 60% by weight of extracted material would be preferable.

Following extraction, the further step of purification of the composition to produce an enriched xanthophyll composition may be performed using techniques well known in the art (for example both extraction of, and means of further purification of pigments are discussed in Strain et al. Biol. Bull. Woods Hole, 86, 169-191, 1944; Strain at al. Phytochemistry 9, 2561-2565, 1970; Hauan and Liaaen-Jensen, Phytochemistry, 28, 2797-2798, 1989). For example, the extracted material may be subjected to column chromatography and fucoxanthin, diatoxanthin and/or diadinoxanthin removed in separate fractions. Purification can be carried out in order to achieve a required standard of purity and/or concentration for the purpose to which the enriched compositions are being put.

Reference to purification of the xanthophyll composition to produce an enriched xanthophyll composition should be taken to mean increasing the amount of fucoxanthin, diatoxanthin and/or diadinoxanthin in the composition in relation to the other components in the composition. Alternatively, decreasing the amount of the other components in the composition in relation to the amount of fucoxanthin, diatoxanthin and/or diadinoxanthin present. An alternative term is “enrichment” of the composition. The amount of fucoxanthin, diatoxanthin and diadinoxanthin as a whole may be increased or alternatively the amount of one or more of fucoxanthin, diatoxanthin and diadinoxanthin in the composition may be increased.

Following purification, the level of fucoxanthin and/or diatoxanthin and/or diadinoxanthin in said enriched composition is preferably at least about 5%, preferably at least about 10%, or more preferably at least about 20%, more preferably at least about 25%, even more preferably at least about 60% by weight of the material in the enriched/purified composition to a maximum of about 85%, preferably 90%, more preferably about 95%, more preferably about 99%, even more preferably substantially 100%. It will be apparent successive purification will result in a higher percentage of xanthophyll in the enriched composition; however levels up to about 30%, more preferably about 50%, more preferably about 70% may still be commercially useful. The preferred ranges may be a combination of any of these upper and lower limits. The total level of fucoxanthin, diatoxanthin and/or diadinoxanthin in the enriched composition may be made up of a mixture of two or three of the compounds or a single compound.

The extraction and purification processes will preferably be undertaken sequentially (when both processes are used). This may occur at the same site, or biomass may be transported to an extraction site and/or the extracted composition to a purification site. Thus the extraction process could be conducted by a party other than that producing the biomass of the invention, as could the purification process. The process of the present invention is intended to include such options.

The xanthophyll compositions and/or enriched xanthophyll compositions containing any one of more of fucoxanthin, diatoxanthin and diadinoxanthin, derived from the process as described, can have therapeutic use in humans or other animals. The xanthophylls fucoxanthin, diatoxanthin and diadinoxanthin have been found to have possible therapeutic and/or cosmetic value. Therapeutic and/or cosmetic uses of such xanthophyll compositions and/or enriched xanthophyll compositions include, but are not limited to, weight-loss, increase resting energy expenditure, anti-carcinogen effects, and anti-inflammatory effects.

The invention provides for use of such xanthophyll compositions and/or enriched xanthophyll compositions in the manufacture of a pharmaceutical or nutraceutical composition for human or animal consumption. Such a composition may be blended with lipids, fatty acids or fatty acid alkyl esters. The compositions may also be blended with plant or seed oil extracts.

The invention provides for xanthophyll compositions and/or enriched xanthophyll compositions produced by the process of the invention, together with a pharmaceutically acceptable excipient, wherein the xanthophyll is selected from any one or more of fucoxanthin, diatoxanthin and diadinoxanthin. Such compositions may optionally include fatty acids and/or fatty acid alkyl esters and plant and/or seed oil extracts. Such compositions can take the form of tablets, capsules or liquid formulations, for example, however any form as would be known to a skilled person could be used.

The invention also provides a process for producing xanthophyll compositions comprising at least one xanthophyll selected from any one or more of fucoxanthin, diatoxanthin and diadinoxanthin, comprising the steps of:
(a) cultivating a microorganism in heterotrophic culture medium;
(b) harvesting cells from the culture medium;
(c) optionally, heat or otherwise killing the cells;
(d) forming the cells into a cake of biomass;
(e) optionally drying the biomass to reduce or eliminate water;
(f) extracting the cake of biomass with a non-selective or selective solvent and recovering the extracted material from the solvent as a residue to produce the xanthophyll composition;
(g) optionally further purifying the fucoxanthin, diatoxanthin and/or diadinoxanthin in said composition to produce an enriched xanthophyll composition.

Current literature identifies methods for commercial exploitation of photosynthetic microalgae for pigment production (for example Jin et al. J. Microbiol. Biotechnol. 13(2): 165-174 2003), but use organisms that do not make Fx, Ddx or Dtx, and therefore do not address the need above.

Furthermore, the means of production of these pigments have relied on photosynthetic or mixotrophic systems where the producing organism can grow under extreme conditions that inhibit the growth of other species (such as extremes of pH or high salinity) in ponds or open raceways (see for example Ben-Amotz, or Cysewski and Lorenz in Handbook of Microalgal Culture, 2004, ed. Richmond) or in photobioreactors supplied internally with large amounts of artificial light and/or comprised of transparent material with high surface to volume ratios to allow light to pass into the culture. The low yields, high capital costs and/or technical difficulties associated with such production and the consequent costs of producing Fx, Ddx or Dtx in this way render them unsuitable for the present purpose.

Jin et al. (J. Microbiol. Biotechnol. 13(2): 165-174, 2003) specifically state that “The most important feature of microalgae is, of course, their photosynthetic ability, which makes them promising organisms for photoautotrophic cultivation on simple mineral media for various biotechnological purposes” thus teaching directly away from the current invention in which heterotrophic cultivation is required.

Zhang in U.S. Pat. No. 7,566,551 provides methods for the production of xanthophylls by microalgae but specifically requires encystment after photosynthetic growth of the organisms thus teaching directly away from the current invention in which heterotrophic cultivation is required.

A large number of authors (reviewed for example by Liaaen-Jensen and Egeland in Chemicals from Microalgae, 1999, ed. Cohen) identify the presence of Fx, Ddx and Dtx in certain groups of microalgae. The presence or absence of these pigments is sometimes used as a tool for identification and classification of the algal specie. These works do not suggest that these algae should be grown for the purpose of providing compositions comprising Fx, Ddx or Dtx nor do they provide methods for doing so at any meaningful scale.

No literature has been identified in which Fx, Ddx or Dtx are identified in microbes grown under heterotrophic conditions and in which it is suggested that these algae should be grown at scale in order to produce a composition comprising the pigment(s).

Surprisingly, the inventors have discovered that Fx, Ddx and Dtx can be produced during heterotrophic growth of microbial strains. Heterotrophic culture has a number of advantages over photosynthetic or mixotrophic growth:
(1) the growth environment can be highly controlled so that pH, temperature and nutrients can be selected for maximum productivity
(2) cells can be grown to much higher density since there is no requirement for light to reach the cells,
(3) culture vessels can relatively easily be sterilised for axenic culture, and
(4) cells thus produced are relatively easy to harvest for product recovery.

Certain microalgae are capable of being grown under heterotrophic conditions. Chen (Trends Biotechnol. 14: 421-426, 1996) teaches that “Heterotrophic culture of microalgae is not without significant problems, including: . . . the inability to produce some light-induced products, such as pigments” thus teaching directly away from the current invention in which heterotrophic cultivation is used to produce pigments.

Vazhappilly and Chen (JAOCS 75: 393-397, 1998) describe heterotrophic growth of a number of algal species but neither record nor mention pigments. The reference neither suggests the processes or compositions described herein, nor that there is any expectation that one could be successful in obtaining such compositions.

Barclay in U.S. Pat. No. 5,130,242 and U.S. Pat. No. 5,908,622 reports the isolation, heterotrophic growth and fatty acid profiles of a number of microalgal strains. The authors concentrate on the strains' abilities to produce fatty acids and mention pigments only in passing, making no reference to Fx, Ddx or Dtx. These works do not suggest that these algae should be grown for the purpose of providing compositions and/or biomass comprising Fx, Ddx or Dtx nor do they provide processes for doing so at any meaningful scale.

Tan and Johns (Journal of Applied Phycology 8: 59-64, 1996) report heterotrophic growth of several diatom species but neither record nor mention pigments. The reference neither suggests the compositions and/or processes described herein, nor that there is any expectation that one could be successful in obtaining such compositions.

Running et al. (Journal of Applied Phycology 6: 99-104,1994) report the production of ascorbic acid by the microalga Chlorella pyrenoidosa by heterotrophic fermentation. The authors do not mention the pigment content of the alga and it would, in any case, not be expected to produce Fx, Ddx or Dtx based on its classification. The authors neither suggest the compositions and/or processes described herein, nor that there is any expectation that one could be successful in obtaining such compositions.

Kitano et al. (Journal of Applied Phycology 9: 559-563, 1997) report heterotrophic growth of the diatom Navicula saprophila but neither record nor mention pigments. The reference neither suggests the compositions and/or processes described herein, nor that there is any expectation that one could be successful in obtaining such compositions.

In U.S. Pat. No. 5,244,921 and U.S. Pat. No. 5,567,732 Kyle and Gladue disclose methods of producing Eicosapentaenoic acid from the diatom Nitzschia alba in heterotrophic culture. The authors state that “the colourless species are especially preferred”, indicating that they perceive the presence of pigments as a disadvantage. The reference does not suggest the compositions and/or processes described herein and instead teaches away from producing pigmented material through heterotrophic growth.

Chu at al. (Journal of Applied Phycology 8: 389-396, 1996) report heterotrophic culture of the diatom Nitzschia inconspicua on acetate and glucose. The authors examine lipid, carbohydrate and protein contents of the cells but do not disclose presence or absence of any pigments. The reference neither suggests the compositions and/or processes described herein, nor that there is any expectation that one could be successful in obtaining such a compositions.

Wen and Chen in a number of papers (Biotechnology Letters 22: 727-733, 2000; Journal of Industrial Microbiology & Biotechnology 25: 218-224, 2000; Enzyme and Microbial Technology 29: 341-347, 2001; Biotechnol. Bioeng. 75: 159-169, 2001; Biotechnol. Prog. 18: 21-28, 2002; Process Biochemistry 37:1447-1453, 2002; Process Biochemistry 38: 523-529, 2002) have described optimisation of heterotrophic growth conditions for the diatom Nitzschia laevis. The authors do not disclose presence or absence of any pigments other than to comment on the brown colour of the cells. The references neither suggest the compositions and/or processes described herein, nor that there is any expectation that one could be successful in obtaining such compositions.

Chu et al. (J. Phycol. 44: 1309-1314, 2008) report heterotrophic culture of the diatom Nitzschia laevis. The authors do not disclose presence or absence of any pigments. The reference neither suggests the compositions and/or processes described herein, nor that there is any expectation that one could be successful in obtaining such compositions.

In Pahl et al. (J Bioscience and Bioeng. 109: 235-239, 2010) the authors report heterotrophic growth of the diatom Cyclotella cryptica for the purposes of producing feed for use in the aquaculture industry. The authors do not disclose presence or absence of any pigments other than to comment on the brown colour of the cells. The reference neither suggests the compositions and/or processes described herein, nor that there is any expectation that one could be successful in obtaining such compositions.

Chen in U.S. Pat. No. 7,063,957 discloses method of production of astaxanthin from the green microalgae Chlorella in heterotrophic culture. The author does not mention the presence of pigments other than astaxanthin, canthaxanthin and adonixanthin and derivates and these microalgae would, in any case, not be expected to produce Fx, Ddx or Dtx based on their classification. The author neither suggests the compositions and/or processes described herein, nor that there is any expectation that one could be successful in obtaining such compositions.

Long in U.S. Pat. No. 6,783,951 provide methods for production of xanthophyll pigments from organisms of the order Thraustochytriales via heterotrophic growth. The author only records the production of β-carotene, astaxanthin, canthaxanthin and adonirubin and these microalgae would, in any case, not be expected to produce Fx, Ddx or Dtx based on their classification. The author neither suggests the compositions and/or processes described herein, nor that there is any expectation that one could be successful in obtaining such compositions.

Griffiths and Geiringer in WO2008004900 describe heterotrophic production of microalgae. The authors mention that, other than the desired fatty acids in the algae, there may be “valuable residual compounds e.g.—sulpho galactolipids, carotenoids, other pigments, amino acids, and other fatty acids capable of serving as foods” but do not provide any expectation as to which particular pigments might be present. The authors therefore neither suggest the compositions and/or processes described herein, nor that there is any expectation that one could be successful in obtaining such compositions.

Furthermore, neither Griffiths and Geiringer, nor any other authors have taught that heterotrophic culture of microalgae can result in production of fucoxanthin, diatoxanthin or diadinoxanthin by the algae such that the result is a composition amenable to use as a source of these compounds.

No other authors have provided methods by which fucoxanthin, diatoxanthin or diadinoxanthin can be obtained from heterotrophically grown microbes.

### EXAMPLES

### Example 1

3 g (dry weight) of a Nitzschia laevis strain In1 culture was transferred into a stirred tank fermenter with a working volume of 15 L. The vessel contained growth media with salts and vitamins together with nutrients at concentrations of: 50 g/L glucose, 2 g/L yeast extract, 6 g/L sodium nitrate, 400 mg/L potassium dihydrogen phosphate and 250 mg/L sodium metasilicate pentahydrate.

Cells were grown in the absence of light.

The culture was aerated with one vessel volume of sterile air per minute and agitation controlled to give a dissolved oxygen content of >50%. pH was maintained at 8 by the addition of sodium metasilicate. Temperature was maintained at 20° C. by the circulation of hot or cold water through a jacket around the fermenter vessel as required.

Sterile samples of biomass were obtained from the culture and material extracted therefrom. Briefly, cells are harvested and washed to remove excess media. The wet equivalent of 0.75 g dry cell weight of cellular material is used to produce cellular extract. Cellular extract containing lipids can be obtained by Folch extraction following the method of Bligh and Dyer (Can. J. Biochem. Physiol. 37: 911-917, 1959).

0.5 μl aliquots of extract were placed on a thin layer chromatography sheet and developed using 25:15:4:2 Chloroform:Methanol:water:acetic acid. Several pigmented bands were observed and these were tentatively identified as carotenes, chlorophyll a, diatoxanthin, diadinoxanthin, fucoxanthin, neofucoxanthin and chlorophyll c based on the work of Strain et al. (Biol. Bull. 86: 169-191, 1944).

Individual pigment bands that had been tentatively identified as diatoxanthin, diadinoxanthin, and fucoxanthin were excised from the TLC plate and redissolved separately in ethanol and acetone and their spectra used to confirm their identity (Table 1).

TABLE 1

| | | Absorbance Peaks | | Absorbance Peaks | |
|---|---|---|---|---|---|
| Band | Identification | in Ethanol | | in Acetone | |
| 2 | Fucoxanthin | 448, 470 | nm | 446, 470 | nm |
| 3 | Diadinoxanthin | (425), 446, 476 | nm | (425), 448, 478 | nm |
| 4 | Diatoxanthin | 451, 480 | nm | (428), 454, 482 | nm |

Note figures in brackets are shoulders rather than true peaks

### Example 2

3 g (dry weight) of a Nitzschia laevis strain In1 culture was transferred into a stirred tank fermenter with a working volume of 15 L. The vessel contained growth media with salts and vitamins together with nutrients at concentrations of: 50 g/L glucose, 2 g/L yeast extract, 6 g/L sodium nitrate, and 400 mg/L potassium dihydrogen phosphate. This represents slight phosphate limitation.

Cells were grown in the absence of light.

The culture was aerated with one vessel volume of sterile air per minute and agitation controlled to give a dissolved oxygen content of >50%. pH was maintained by the addition of sodium metasilicate. Temperature was maintained at 20° C. by the circulation of hot or cold water through a jacket around the fermenter vessel as required.

Once the culture reached high cell density, harvest was carried out at 6 hourly intervals. Harvested volume was replaced with fresh media to produce a continuous culture.

Harvested biomass was collected and centrifuged. The cake of biomass was extracted using near-critical dimethyl ether as a solvent and the resulting extract containing the lipids and pigments from the biomass was examined for fucoxanthin content by HPLC. The fucoxanthin quantification was done by constructing a calibration curve for a pure fucoxanthin standard (Sigma Aldrich), and then comparing the mass or concentration in the sample with the calibration curve. Extract was recovered as 11% of dry cell weight and the fucoxanthin content of the extract was determined to be 17.9 mg/g. Combined these give a fucoxanthin content of 0.2% of dry cell weight.

### Example 3

A culture of Nitzschia laevis was grown in an airlift fermenter with a working volume of 500 L. The culture was grown in a semi-continuous manner in which 250 L of the culture was harvested once every 24 hours and the volume returned to 500 L with growth media comprising salts and vitamins together with nutrients at concentrations of: 75 g/L glucose, 3 g/L yeast extract, 9.9 g/L sodium nitrate, and 920 mg/L disodium hydrogen phosphate.

Sodium metasilicate was also supplied to the culture on a regular, periodic basis to provide a silica source for the cells. In this manner cultures could be maintained in which the cells were at above 20 g dry cell weight per litre at the time of harvest. At the time of harvest the contents of glucose, nitrate and phosphate in the culture were all determined to be greater than zero and so these nutrients were present in non-limiting amounts through the growth period.

Cells were grown in the absence of light.

The culture was aerated with a volume of sterile air per minute sufficient to maintain a dissolved oxygen content of >40%. pH was maintained between 7.8 and 8.2 by the addition of sodium hydroxide. Temperature was maintained at 18° C. by the continuous circulation of water at this temperature through a jacket around the fermenter vessel.

Culture drawn off the main vessel was subjected to centrifugation as a means of collecting biomass. The biomass was then dried by freeze drying. The biomass was extracted with 85:15 isopropanol:water solvent mixture and the solvent was removed by vacuum assisted evaporation. The extract was recovered as 8.2% of dry cell weight.

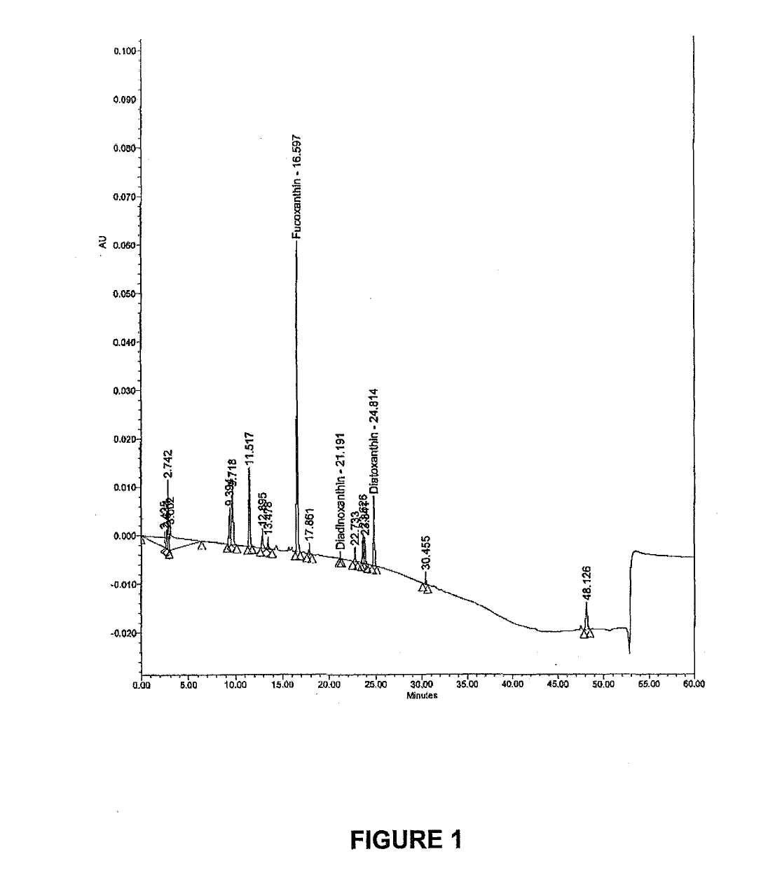

A typical HPLC spectrum of the extract recorded at 449 nm is shown in FIG. 1. Pigment identification was performed by comparison of elution times against a commercially obtained pigment standard and confirmed using mass spectroscopy. The presence of fucoxanthin, diatoxanthin and diadinoxanthin in the extract was confirmed.

Quantification of the content of fucoxanthin, diatoxanthin and diadinoxanthin was performed using commercially obtained purified fucoxanthin standard and extinction coefficients for fucoxanthin and diatoxanthin. Contents of the three pigments in the extract and a calculated content in dry biomass are shown in Table 2.

TABLE 2

| | Content in Extract | Proportion of dry biomass |
|---|---|---|
| Pigment | (mg/g) | (% DCW) |
| Fucoxanthin | 157.7 | 1.29 |
| Diatoxanthin | 25.4 | 0.21 |
| Diadinoxanthin | 0.3 | 0.002 |
| Total | 183.4 | 1.50 |

At least 10 g dry weight of cells is being harvested per litre of culture per day in this process. At 1.5% of dry cell weight, the total production of Fx, Dtx and Ddx is at least 6 mg/L/hour. Individually, Fx makes up 1.29% of dry cell weight and productivity of this pigment in isolation is at least 5.3 mg/L/hour.

### Example 4

A culture of Nitzschia laevis was grown in an airlift fermenter with a working volume of 500 L. The culture was grown in a semi-continuous manner in which 250 L of the culture was harvested once every 24 hours and the volume returned to 500 L with growth media comprising salts and vitamins together with nutrients at concentrations of: 75 g/L glucose, 3 g/L yeast extract, 9.9 g/L sodium nitrate, and 920 mg/L disodium hydrogen phosphate.

Sodium metasilicate was also supplied to the culture on a regular, periodic basis to provide a silica source for the cells. In this manner cultures could be maintained in which the cells were at above 20 g dry cell weight per litre at the time of harvest. At the time of harvest the contents of glucose, nitrate and phosphate in the culture were all determined to be greater than zero and so these nutrients were present in non-limiting amounts through the growth period.

Cells were grown in the absence of light.

The culture was aerated with a volume of sterile air per minute sufficient to maintain a dissolved oxygen content of >40%. pH was maintained between 7.8 and 8.2 by the addition of sodium hydroxide. Temperature was maintained at 18° C. by the continuous circulation of water at this temperature through a jacket around the fermenter vessel.

Culture drawn off the main vessel was subjected to centrifugation as a means of collecting biomass. A small sample was taken and the water content of the biomass paste was determined to be 82% by weight. Four volumes wet biomass paste was then mixed with 1 volume water to form a slurry and frozen at −20° C.

The frozen biomass was defrosted and 200 mL mixed with 300 mL ice-cold acetone. This mixture was subject to gentle agitation for two hours.

The acetone-water, now containing pigments, was separated from the remaining solid matter by filtration. The solids were then washed with an additional 100 mL ice-cold acetone. 375 mL of filtrate was collected, 100 mL of which was dried under vacuum to give an extract.

A further 100 mL was placed at −20° C. for 72 hours. During this time, a dark green precipitate formed. The supernatant was decanted and dried under vacuum.

Quantification of the content of fucoxanthin, diatoxanthin and diadinoxanthin was performed using commercially obtained purified fucoxanthin standard and extinction coefficients for fucoxanthin and diatoxanthin. Contents of the fucoxanthin and diatoxanthin in the extract are shown in Table 3.

TABLE 3

| | Content in Acetone | Content in Frozen Acetone |
|---|---|---|
| Pigment | Extract (mg/g) | Extract Supernatant (mg/g) |
| Fucoxanthin | 350 | 640 |
| Diatoxanthin | 50 | 70 |

### Example 5

Isochrysis galbana, an alga of the class Prymnesiophyceae, is cultured heterotrophically under conditions chosen to produce biomass comprising the xanthophyll pigments Fucoxanthin, Diatoxanthin and Diadinoxanthin at levels greater than about 0.2% of dry cell weight. The biomass is harvested and an extraction with solvents is performed on the harvested biomass. The extract is then further purified to produce a xanthophyll composition in which fucoxanthin, diatoxanthin and diadinoxanthin combined form at least 30% of the total weight of the composition. This purified composition is then further purified to produce separate compositions comprising each of fucoxanthin, diatoxanthin and diadinoxanthin at over 90% purity.

### Example 6

Screens are carried out to identify members of the classes Diatomophyceae, Prymnesiophyceae and Pelagophyceae that are capable of heterotrophic growth and in which xanthophyll pigments are found at levels greater than about 0.5% of dry cell weight. The microorganism is then grown under heterotrophic conditions chosen to maximise the production of one or more of these xanthophyll pigments. The biomass is harvested and an extraction with solvents is performed on the harvested biomass. The extract is then further purified to produce a xanthophyll composition in which fucoxanthin, diatoxanthin and diadinoxanthin combined form at least 30% of the total weight of the composition. This purified composition is then further purified to produce separate compositions comprising each of fucoxanthin, diatoxanthin and diadinoxanthin at over 90% purity.

### Example 7

Cyclotella cryptica, a diatom, is cultured heterotrophically under conditions chosen to produce biomass comprising the xanthophyll pigments Fucoxanthin, Diatoxanthin and Diadinoxanthin. The biomass is harvested and an extraction with solvents is performed on the harvested biomass. The extract is then further purified to produce a xanthophyll composition in which fucoxanthin, diatoxanthin and diadinoxanthin combined form at least 30% of the total weight of the composition. This purified composition is then further purified to produce separate compositions comprising each of fucoxanthin, diatoxanthin and diadinoxanthin at over 90% purity.

### Example 8

Phaeodactylum tricomutum, a diatom that is an obligate photoautotroph, is transformed according to the method of Apt et al. (U.S. Pat. No. 7,939,710) to give it the ability to grow heterotrophically using glucose as a source of energy and carbon. The transformed organism is cultured heterotrophically under conditions chosen to produce biomass comprising the xanthophyll pigments Fucoxanthin, Diatoxanthin and Diadinoxanthin. The biomass is harvested and an extraction with solvents is performed on the harvested biomass. The extract is then further purified to produce a xanthophyll composition in which fucoxanthin, diatoxanthin and diadinoxanthin combined form at least 30% of the total weight of the composition. This purified composition is then further purified to produce separate compositions comprising each of fucoxanthin, diatoxanthin and diadinoxanthin at over 90% purity.

### General

Example 1 shows a process for producing a microbial biomass containing diatoxanthin, diadinoxanthin and fucoxanthin comprising the steps of cultivating a microorganism in heterotrophic culture, recovering the biomass and extracting the biomass to recover a composition containing Fucoxanthin, Diadinoxantin and Diatoxanthin. Purification and separation by chromatography is also demonstrated.

Examples 2 and 3 show a process for producing a microbial biomass with a fucoxanthin content of 0.2% of dry cell weight comprising the steps of cultivating a microorganism in heterotrophic culture and recovering the biomass. The example also shows extraction of the biomass to produce a xanthophyll composition. Example 3 further shows purification, separation and quantification of Fucoxanthin, Diatoxanthin and Diadinoxanthin via HPLC.

The inventors expect that on optimization of the conditions of the heterotrophic culture the content of diatoxanthin, diadinoxanthin and/or fucoxanthin will increase.

Example 2 also shows a process for producing a fucoxanthin containing composition derived from the process of the invention, comprising the steps of, harvesting cells from the culture medium, forming the cells into a cake of biomass, extracting the biomass with a non-selective solvent, recovering the extracted material and separating fucoxanthin by HPLC.

Example 3 shows a process for producing the xanthophyll pigments at a rate greater than 6 mg per litre of culture per hour. The inventors expect that optimisation of the conditions of the heterotrophic culture will increase the rate of production of these pigments.

Example 4 shows a process for producing xanthophyll compositions through the use of selective solvents and enriching the xanthophyll content of such compositions through a simple purification step. As can be seen from this Example levels of xanthophyll greater than 40% by weight of the extract can be achieved. it is therefore envisioned that levels of at least 60% could be achieved.

Examples 5-8 demonstrate that levels of greater than 90% xanthophyll by weight of an enriched composition can be achieved following purification.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention defined in the attached claims.

1. A microbial biomass, produced from a heterotrophic fermentation, the biomass comprising at least one xanthophyll selected from any one or more of fucoxanthin, diatoxanthin and diadinoxanthin.

2. The microbial biomass of claim 1 wherein the xanthophyll is fucoxanthin.

3-5. (canceled)

6. The microbial biomass of claim 1 wherein the xanthophyll is present at levels equal to or greater than about 0.1% of dry cell weight of the biomass.

7-9. (canceled)

10. The microbial biomass of claim 1 wherein the microbial biomass is a marine diatom biomass.

11. (canceled)

12. A process for producing a microbial biomass, wherein the microbial biomass comprises at least one xanthophyll selected from any one or more of fucoxanthin, diatoxanthin and diadinoxanthin, the process comprising the steps of:
cultivating a microorganism in heterotrophic culture to produce the biomass; and
recovering said biomass.

13. The process of claim 12 wherein the xanthophyll is fucoxanthin.

14-15. (canceled)

16. The process of claim 12 wherein the xanthophyll is present at levels equal to or greater than about 0.1% of dry cell weight of the biomass.

17-19. (canceled)

20. The process of claim 12 wherein the microbial biomass is a marine diatom biomass.

21. (canceled)

22. The process of claim 12 wherein at least about 1 mg of the xanthophyll is produced per litre of culture per hour.

23. (canceled)

24. The process of claim 12 wherein the step of cultivating the microorganism in heterotrophic culture comprises a culture phase in which cells are grown under conditions in which organic carbon is used as an energy source.

25. The process of claim 12 wherein the step of cultivating the microorganism in heterotrophic culture comprises a culture phase in which cells are grown under conditions of limitation of nutrients.

26. (canceled)

27. The process of claim 12 wherein the step of cultivating a microorganism in heterotrophic culture comprises exposing the culture to low levels of light.

28-29. (canceled)

30. The process of claim 12 wherein the step of cultivating a microorganism is carried out as a continuous fermentation.

31. The process of claim 12 wherein the step of cultivating a microorganism in heterotrophic culture comprises two stages;
(i) a growth phase in which cells are grown under conditions in which organic carbon is used as an energy source and other nutrients are not limiting, said first step being undertaken to accumulate biomass, and
(ii) a finishing phase in which cells are grown under alternative conditions from the first stage.

32. The process of claim 31 wherein the alternative conditions in stage (ii) are selected from any one of more of:
limitation of nutrients,
changes in pH,
changes in temperature,
changes in salinity and/or
exposure to low levels of light energy,
said conditions being undertaken in order to maximise the amount of recoverable xanthophyll in the biomass.

33-36. (canceled)

37. A microbial biomass produced by the process of claim 12, the biomass comprising at least one xanthophyll selected from any one or more of fucoxanthin, diatoxanthin and diadinoxanthin.

38. A xanthophyll composition comprising at least one xanthophyll selected from any one or more of fucoxanthin, diatoxanthin and diadinoxanthin, wherein the composition is extracted from a microbial biomass produced from a heterotrophic fermentation.

39. The xanthophyll composition of claim 38 wherein the composition is extracted from the microbial biomass by selective or non-selective extraction.

40. The xanthophyll composition of claim 38 wherein the xanthophyll is present at levels of about 0.1 to 60% by weight.

41. (canceled)

42. An enriched xanthophyll composition comprising at least one xanthophyll selected from any one or more of fucoxanthin, diatoxanthin and diadinoxanthin, wherein the composition is extracted from a microbial biomass produced from a heterotrophic fermentation and purified.

43. (canceled)

44. The enriched xanthophyll composition of claim 42 wherein the xanthophyll is present at levels of at least about 25% by weight.

45. The enriched xanthophyll composition of claim 42 wherein the xanthophyll is present at levels of at least about 60% by weight.

46. A process for producing a xanthophyll composition, wherein the xanthophyll is selected from any one or more of fucoxanthin, diatoxanthin and diadinoxanthin, comprising the steps of:
cultivating a microorganism in heterotrophic culture to produce a biomass;
recovering said biomass; and
recovering said xanthophyll composition from said biomass.

47. The process of claim 46 wherein the xanthophyll is fucoxanthin.

48-50. (canceled)

51. The process of claim 46 wherein the microbial biomass is a marine diatom biomass.

52. (canceled)

53. The process of claim 46 wherein at least about 1 mg of the xanthophyll is produced per litre of culture per hour.

54-56. (canceled)
